(19) United States
(12) Patent Application Publication
(10) Pub. No.: US 2022/0189334 A1
(43) Pub. Date: Jun. 16, 2022

(54) COGNITION EVALUATION SYSTEM AND METHOD

(71) Applicant: NATIONAL CHENG KUNG UNIVERSITY, Tainan City (TW)

(72) Inventors: Fu-Zen SHAW, Tainan City (TW); Sheng-Fu LIANG, Tainan City (TW); Ren-Yi LIN, Tainan City (TW)

(73) Assignee: NATIONAL CHENG KUNG UNIVERSITY, Tainan City (TW)

(21) Appl. No.: 17/166,907

(22) Filed: Feb. 3, 2021

(30) Foreign Application Priority Data
Dec. 11, 2020 (TW) 109143949

(51) Int. Cl. G09B 19/00; A61B 5/055; A61B 5/245; A61B 5/369; A61B 5/00; A61B 6/00; A61B 5/16

(52) U.S. Cl. CPC G09B 19/00; A61B 5/055; A61B 5/245; A61B 5/369; A61B 5/0075; A61B 2505/09; A61B 5/0042; A61B 5/486; A61B 5/165; A61B 2503/10; A61B 6/501

(57) ABSTRACT

A cognition evaluation method, comprises: obtaining brain physiology information associated with a subject by a brain state measuring instrument, obtaining a plurality of cognitive aspects according to the brain physiology information, selecting at least one of the cognitive aspects according to a sport type as at least one sport cognitive aspect, obtaining a cognition task outcome corresponding to the at least one sport cognitive aspect, and evaluating and outputting a sport cognition level of the at least one sport cognitive aspect according to the cognition task outcome. The present disclosure further provides a cognition evaluation system.

### CROSS-REFERENCE TO RELATED APPLICATIONS

This non-provisional application claims priority under 35 U.S.C. § 119(a) on Patent Application No(s). 109143949 filed in Republic of China (ROC) on Dec. 11, 2020, the entire contents of which are hereby incorporated by reference.

### BACKGROUND

### 1. Technical Field

This disclosure relates to a cognition evaluation system and method.

### 2. Related Art

Cognitive function is the ability of a human brain on processing, memorizing and accessing information. That is, cognitive function indicates the ability of people grasping the composition of things, the relationship between performance and other related things, the driving force of development, the direction of development and basic regularities. Many veteran athletes can still win the games using on their experiences and skills even when their physical abilities (muscular power, muscular endurance or coordination) can't be maintained at their peaks, and it's mostly relied on the comprehensive performance of their cognitive functions. These cognitive functions may include the dynamic vision, selective attention, working memory, dynamic objective planning, and peripheral vision of an athlete. In the past, these cognitive functions are called “gift”.

For the measurement of physical fitness, modern sports science has proposed a number of effective measurement, evaluation and training methods. For example, electromyography, electrocardiogram, vital capacity and blood screening are used to measure physical fitness. Other methods include evaluating a player's skills through video recording and motion analyzer. However, many professional sports are still exploring the field of cognition function. Most of them use the correctness of the execution of simulation training related to a sport to evaluate an athlete's ability, and rarely use the changes in brain state of the athlete when performing tasks to evaluate the athlete's ability. Therefore, the evaluation of an athlete's overall sports cognitive function or the providing of suitable training sessions to an athlete still relies on the past experience of the trainer or coach.

Moreover, determining the cognitive function of athletes by measuring the brain signals is usually achieved through simulation training. However, simulation training often involves various cognitive functions, and different sport often adopts different ways of measuring the brain signals. The lack of analogy makes it difficult to accurately measure brain responses.

### SUMMARY

Accordingly, this disclosure provides a cognition evaluation system and method.

According to one or more embodiment of this disclosure, a cognition evaluation method, comprising: obtaining brain physiology information associated with a subject by a brain state measuring instrument; obtaining a plurality of cognitive aspects according to the brain physiology information; selecting at least one of the cognitive aspects according to a sport type as at least one sport cognitive aspect; obtaining a cognition task outcome corresponding to the at least one sport cognitive aspect; and evaluating and outputting a sport cognition level of the at least one sport cognitive aspect according to the cognition task outcome.

According to one or more embodiment of this disclosure, a cognition evaluation system, comprising: a brain state measuring instrument, configured to obtain brain physiology information associated with a subject; and a computing device, configured to obtain a plurality of cognitive aspects according to the brain physiology information, and select at least one of the cognitive aspects according to a sport type as at least one sport cognitive aspect, the computing device configured to obtain a cognition task outcome corresponding to the at least one sport cognitive aspect, and further evaluate and outputs a sport cognition level of the at least one sport cognitive aspect according to the cognition task outcome.

### BRIEF DESCRIPTION OF THE DRAWINGS

The present disclosure will become more fully understood from the detailed description given hereinbelow and the accompanying drawings which are given by way of illustration only and thus are not limitative of the present disclosure and wherein:

FIG. 1 is a block diagram illustrating a cognition evaluation system according to an embodiment of the present disclosure;

FIG. 2 is a flow chart illustrating a cognition evaluation method according to an embodiment of the present disclosure;

FIG. 3 is a flow chart illustrating a cognition evaluation method according to another embodiment of the present disclosure;

FIG. 4 is an exemplary diagram illustrating a normal distribution model for evaluating a sport cognition level according to an embodiment of the present disclosure; and

FIG. 5 is a flow chart illustrating a cognition evaluation method according to yet another embodiment of the present disclosure.

### DETAILED DESCRIPTION

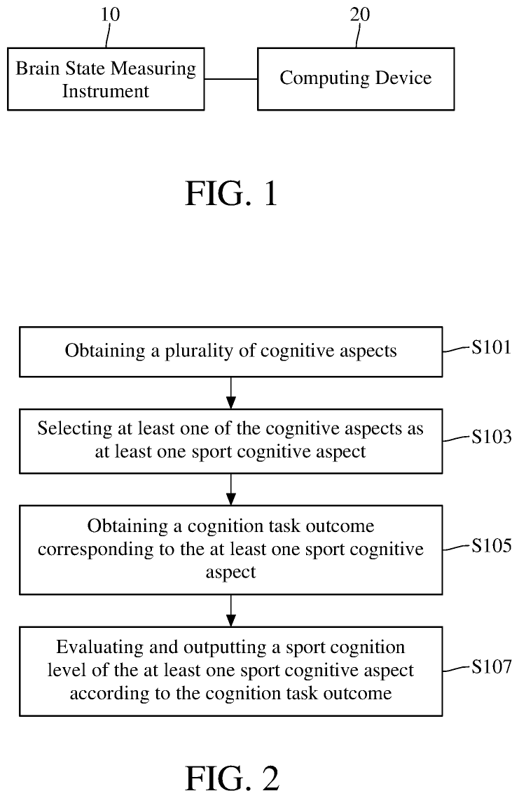

Please refer to FIG. 1 which is a block diagram illustrating a cognition evaluation system according to an embodiment of the present disclosure. The cognition evaluation system provided by the present disclosure preferably comprises a brain state measuring instrument 10 and a computing device 20. The brain state measuring instrument 10 is configured to obtain brain physiology information associated with a subject. The brain state measuring instrument 10 is in signal-transmittable connection with the computing device 20, for the computing device 20 to analyze the brain physiology information obtained by the brain state measuring instrument 10 and to further output a sport cognition level corresponding to the subject. The brain physiology information is formed by the physiological activity and level of development of the subject's brain. The brain physiology information includes, but not limited to, temporal signals such as brain waves, blood oxygen concentration, magnetic flux, and spatial information such as gray and white matter distribution, neural connections, and brain structure.

The brain state measuring instrument 10 can be a measuring instrument using structural magnetic resonance imaging (sMRI), functional magnetic resonance imaging (fMRI), magnetoencephalography (MEG), electroencephalography (EEG), near infrared spectroscopy (NIRS), X-ray, etc; the computing device 20 can be a server, a central processor or other device with computing capabilities, the present disclosure does not limit the types of the brain state measuring instrument 10 and the computing device 20.

Please refer to both FIGS. 1 and 2, wherein FIG. 2 is a flow chart illustrating a cognition evaluation method according to an embodiment of the present disclosure.

Step S101: Obtaining a plurality of cognitive aspects.

The brain state measuring instrument 10 is used to measure the physiological information of the brain during the execution of a certain task (for example, running, swinging, pitching, etc.) of a subject to capture brain wave characteristics. Accordingly, the computing device 20 can determine a plurality of cognitive aspects associated with the subject based on the brain physiological information.

In detail, the computing device 20 can evaluate the brain physiological information obtained by the brain state measuring instrument 10 to obtain the cognitive aspects, wherein the computing device 20 evaluates the brain physiological information can be a functional evaluation of brain waves or brain images. For example, the functional evaluations can be large scale brain network evaluation, inter-area connectivity evaluation, task-related pathway evaluation, etc. The large scale brain network can further include default mode network, dorsal attention network, ventral attention network, salience network, frontal-parietal network (FPN), visual network and limbic network, but the present disclosure is not limited thereto. Moreover, the computing device 20 can perform structural evaluation on the brain physiological information, such as the gray and white matter evaluation, or perform evaluation on diffusion-tensor imaging (DTI), diffusion weight imaging (DWI) to determine the degree of neural connections, but the present disclosure is not limited thereto.

In addition, the cognitive aspects can comprise one or more of an inhibition control aspect, a visual perception aspect, a visual attention aspect, a memory aspect, an emotion aspect and a semantics aspect. The cognitive task corresponding to the inhibition control aspect can be a go-on-go task or a stop signal task; the cognitive task corresponding to the visual perception aspect can be a coherence motion task or a motion suppression; the cognitive task corresponding to the visual attention aspect can be an attention network task (ANT), a gaze cueing task, a multiple object tracking task, or a reaction time task; the cognitive task corresponding to the memory aspect can be a backward digit span task or a N-back task; the cognitive task corresponding to the emotion aspect can be a perceived stress scale (PSS) or an emotional Stroop task; the cognitive task corresponding to the semantics aspect can be semantics training on different sports, such as visual-motor behavior, symbolic learning theory or psycho-neuromuscular theory. However, the abovementioned cognitive aspects and cognitive tasks are preferred embodiments, the present disclosure does not limit the types of cognitive aspects and cognitive tasks.

Step S103: Selecting at least one of the cognitive aspects as at least one sport cognitive aspect.

The computing device 20 selects at least one of the cognitive aspects according to a sport type from the plurality of cognitive aspects as at least one sport cognitive aspect. For example, when the sport type is baseball, the computing device 20 can select the inhibition control aspect, the visual attention aspect, the visual perception aspect, and the semantics aspect from the plurality of cognitive aspects as the sport cognitive aspect. Regardless of the sport type, the sport cognitive aspect preferably includes both the inhibition control aspect and the semantics aspect. Specifically, when engaging in various sports, there will be situations where the athlete has to organize, understand, and process (read) the information regarding the sport then decide make action or not, in a short period of time. Therefore, measuring the inhibitory control aspect and semantic aspect of the athlete can be used to evaluate the ability of decision making and comprehension. Take baseball as an example, a batter tries to suddenly break his swing because the coming pitch-ball is out of hit-zone or hot-zone. The ability of the batter's inhibition control aspect greatly affects whether the player can interrupt the swing action within tens of milliseconds after perceiving the ball. In addition, the ability of semantics aspect can be understood as the ability of players to how quickly access the information of strategy and tactics. That information including, but not limited to, the coach's signs, base-running gestures, the shifting of the opponent's defensive lineup, and other implications.

Step S105: Obtaining a cognition task outcome corresponding to the at least one sport cognitive aspect.

The cognition task outcome can be the quantitative data in the sport cognitive aspect. In other words, each obtained sport cognitive aspect includes a quantitative data such as the degree of completion of the task, degree of correctness of the task, execution time of the task and the number of completion times. The computing device 20 uses the quantitative data obtained based on each sport cognitive aspect as the cognition task outcome corresponding to the sport cognitive aspect, wherein the quantitative data that each sport cognitive aspect corresponds to can be one or more quantitative data, the present disclosure is not limited thereto. In addition, the cognition task outcome can further comprise the physiological states measured by the brain state measuring instrument 10, such as neural connection, blood oxygen concentration, or gray and white matter distribution.

Step S107: Evaluating and outputting a sport cognition level of the at least one sport cognitive aspect according to the cognition task outcome.

As described above, the cognition task outcome can be the quantitative data which each sport cognitive aspect corresponds to. Therefore, the computing device 20 can evaluate the cognition task outcome to output the sport cognition level that the sport cognitive aspect corresponds to. Namely, the sport cognition level is for representing the sport performance of the subject at the corresponding sport cognitive aspect.

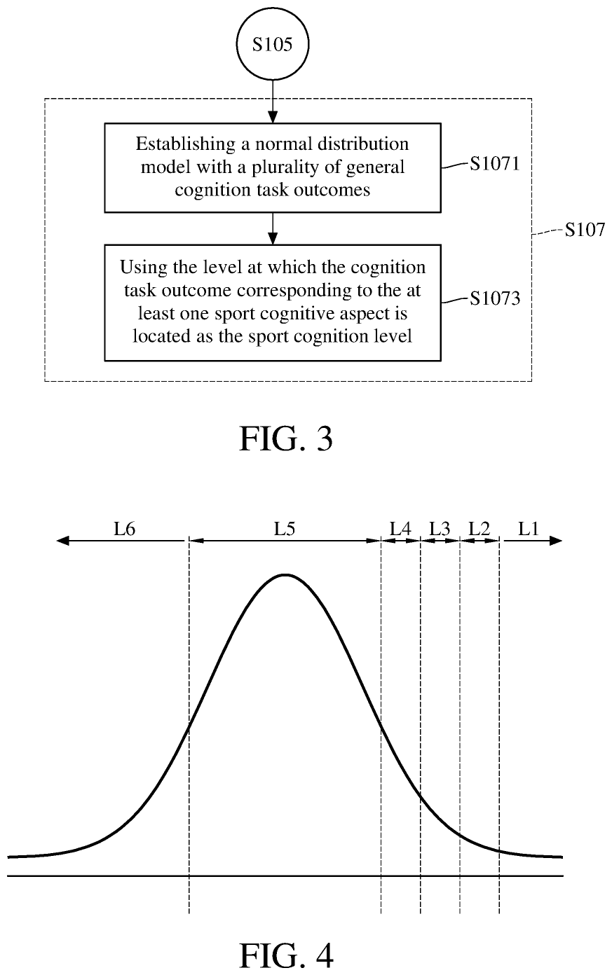

Please refer to FIGS. 1, 3 and 4, wherein FIG. 3 is a flow chart illustrating a cognition evaluation method according to another embodiment of the present disclosure; FIG. 4 is an exemplary diagram illustrating a normal distribution model for evaluating a sport cognition level according to an embodiment of the present disclosure.

After obtaining the cognition task outcome corresponding to the sport cognitive aspect in step S105, evaluating and outputting the sport cognition level of the sport cognitive aspect according to the cognition task outcome described in step S107 can be implemented as steps S1071 and S1073 as shown in FIG. 3.

Step S1071: Establishing a normal distribution model with a plurality of general cognition task outcomes.

In detail, before evaluating the sport cognition level of a certain subject, the computing device 20 can first obtain a plurality of general cognition task outcomes, and use the plurality of general cognition task outcomes to establish a normal distribution model as shown in FIG. 4. For example, the certain subject is a 22-year-old professional track and field athlete, and the plurality of general cognition task outcomes is the cognition task outcomes of a plurality of ordinary 22-year-old people performing the same cognitive task (track and field). The plurality of general cognition task outcomes can be used for the computing device 20 to determine the sport cognition level of the 22-year-old professional track and field athlete based on the general cognition task outcomes of the general public. Therefore, the computing device 20 can establish a normal distribution model based on the cognition task outcomes of ordinary 22-year-old people performing the cognitive task related to track and field. Further, in addition to the ordinary 22-year-old people, the general cognition task outcomes can also include the cognition task outcomes of other subjects who are also professional track and field athletes and of the same age, or only includes the cognition task outcomes of other subjects of the same age who are also professional track and field athletes. Accordingly, the normal distribution model corresponding to the sport can be obtained subsequently for the sport that the certain subject is participating in.

Also, the cognition evaluation system can further comprise a cognition task outcome database for storing a plurality of cognition task outcomes of a plurality of athletes, and another cognition task outcome database for storing all the cognition task outcomes except the cognition task outcomes of semantics aspect of the athletes. Accordingly, the cognition task outcomes stored in the another cognition task outcome database can be used for subsequent horizontal evaluation. In other words, if the athlete is a boxer, then the ability of the athlete (boxer) to engage in baseball can be evaluated based on the cognition task outcomes stored in the another cognition task outcome database.

Step S1073: Using the level at which the cognition task outcome corresponding to the at least one sport cognitive aspect is located as the sport cognition level.

The normal distribution model can have a plurality of value domains, and each of the value domain corresponds to a level, such as the first level L1 to the sixth level L6 shown in FIG. 4. Therefore, the computing device 20 can determine which level, among first level L1 to sixth level L6, that the cognition task outcome corresponding to the sport cognitive aspect falls within, and use the level that the cognition task outcome is located as the sport cognition level. Accordingly, the computing device 20 can output the sport cognition level, so that the subject, trainer and/or coach can determine the ability of the subject's sport cognition ability relative to the general public or athletes of the same type.

For example, the first level L1 can be the elite level; the second level L2 can be the professional level; the third level L3 can be the amateur level; the fourth level L4 can be the rookie level; the fifth level L5 can be the general public level; the sixth level L6 can be the level representing people who aren't suitable for performing the sport corresponding to the normal distribution model. In other words, when the sport cognition level is the first level L1 to the fourth level L4, it means that the athlete has the potential in that sport.

In addition, after determining the sport cognition level, the computing device 20 can further update the boundary value of the level that the cognition task outcome is located at (the sport cognition level) using the cognition task outcome, so that each level can be more in line with the corresponding cognition task outcome. In detail, the computing device 20 incorporates the cognition task outcome into the normal distribution model, so that the normal distribution curve and the boundary value of the level of the model are updated.

By using the normal distribution model, the determined sport cognition level can be the level that best matches the subject. In other words, since the normal distribution model is established based on different ages, genders, races, sports type etc., a suitable normal distribution model can be used respectively on each subject to accurately determine the sport cognition level of each subject.

It should be noted that the first level L1 to the fourth level L4 shown in FIG. 4 have the same width on their horizontal axis, and the fifth level L5 and the sixth level L6 have shown in FIG. 4 have different width on their horizontal axis, but the present disclosure is not limited thereto. Besides, after updating the boundary value of the corresponding level as described above, the widths of the corresponding level and other level(s) adjacent thereto are also changed.

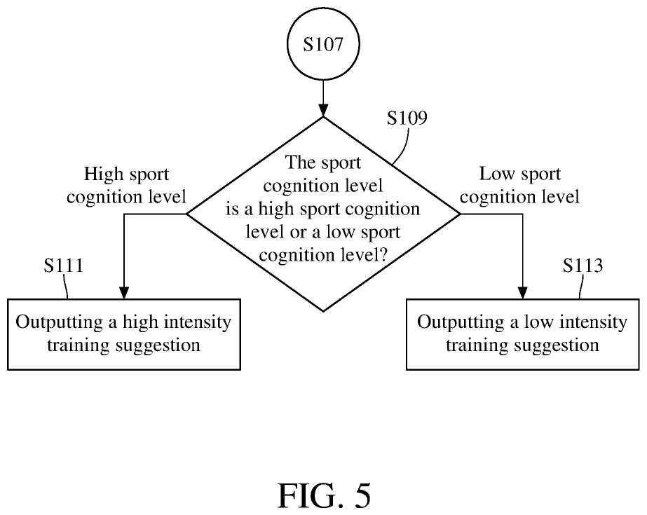

Please refer to both FIGS. 4 and 5, wherein FIG. 5 is a flow chart illustrating a cognition evaluation method according to yet another embodiment of the present disclosure.

Step S109: Determining whether the sport cognition level is a high sport cognition level or a low sport cognition level.

After the level that the cognition task outcome is located at is determined, the computing device 20 can further determine if the cognition task outcome is located at a high sport cognition level or a low sport cognition level.

For example, when the general cognition task outcomes used for establishing the normal distribution model are the cognition task outcomes of the general public, then the high sport cognition level is the first level L1 shown in FIG. 4, and the low sport cognition level is, for example, the second level L2 shown in FIG. 4. When the general cognition task outcomes used for establishing the normal distribution model are the cognition task outcomes of athletes of the same type of sport, then the high sport cognition levels are, for example, the first level L1 and second level L2 shown in FIG. 4, and the low sport cognition levels are, for example, the fifth level L5 and the sixth level L6 shown in FIG. 4. However, the levels representing the high/low sport cognition levels described are merely examples, the present disclosure is not limited thereto.

When the computing device 20 determines the sport cognition level is the high sport cognition level, the computing device 20 can perform step S111: outputting a high intensity training suggestion. On the other hand, when the computing device 20 determines the sport cognition level is the low sport cognition level, the computing device 20 can perform step S113: outputting a low intensity training suggestion.

Specifically, when the computing device 20 determines the sport cognition level is the high sport cognition level, it means the subject performs well on that type of sport. Therefore, the computing device 20 can output the high intensity training suggestion accordingly, to suggest the subject, trainer and/or coach to arrange a high intensity training session that matches the subject's sport cognition level. On the other hand, when the computing device 20 determines the sport cognition level is the low sport cognition level, it means the subject's performance isn't ideal enough. Therefore, the computing device 20 can output the low high intensity training suggestion accordingly, to suggest the subject, trainer and/or coach to arrange a low intensity training session that matches the subject's sport cognition level. The low intensity training suggestion includes, for example, suggestion of phase training, suggestion of lowering the intensity of training and increasing the amount of training.

Please continue referring to FIG. 4, in addition to the description above, the computing device 20 can further adjust the boundary value of the high sport cognition level of the normal distribution model according to a sport scale (sports event). For example, when the subject is entering Olympics, the computing device 20 can increase the lower boundary value of the high sport cognition level; when the subject is entering the Universiade held by the International University Sports Federation (FISU), the computing device 20 can lower the lower boundary value of the high sport cognition level, so that the training suggestion outputted subsequently can be more in line with the level of the sports event.

The present disclosure can be used in different scenarios. For example, when the subject is an athlete who has been injured, the cognition evaluation system and method of the present disclosure can be used to determine the athlete's sport cognition level to further determine the athlete's brain structure and physical connectivity to evaluate the athlete's recovery of cognitive function, and provide a training session that is more suitable for the athlete accordingly. Similarly, when the subject is a teenager, since the subject's brain structure may change as the subject ages, the cognition evaluation system and method of the present disclosure can be used to determine the subject's sport cognition level as well as the long-term plasticity of the cognitive function of the subject.

In summary of the description of the cognition evaluation system and method above, taking the subject who is a table tennis player as an example, the sport cognitive aspects of the subject at least include the inhibition control aspect, the visual attention aspect, the emotion aspect and the semantics aspect, wherein the semantics aspect is related to the subject's ability to predict the placement of the ball. When the subject is executing the cognitive task(s) corresponding to table tennis, the brain state measuring instrument 10 obtains brain physiology information associated with the subject. Therefore, when the subject finishes the cognitive task(s), the computing device 20 is able to evaluate and output a corresponding sport cognition level according to the internal data (each cognitive aspect) and external data (for example, the connectivity of the brain) of the cognition task outcome.

In view of the above description, according to the cognition evaluation system and method of the present disclosure, the cognitive function of an athlete may be effectively evaluated. The outcomes may be integrated with multiple cognition task outcome to generate graphs, tables, and symbols. So that the trainer or coach may arrange a training session for the athlete according to the athlete's cognitive function.

The present disclosure has been disclosed above in the embodiments described above, however it is not intended to limit the present disclosure. It is within the scope of the present disclosure to be modified without deviating from the essence and scope of it. It is intended that the scope of the present disclosure is defined by the following claims and their equivalents.

What is claimed is:

1. A cognition evaluation method, comprising:
obtaining brain physiology information associated with a subject by a brain state measuring instrument;
obtaining a plurality of cognitive aspects according to the brain physiology information;
selecting at least one of the cognitive aspects according to a sport type as at least one sport cognitive aspect;
obtaining a cognition task outcome corresponding to the at least one sport cognitive aspect; and
evaluating and outputting a sport cognition level of the at least one sport cognitive aspect according to the cognition task outcome.

2. The cognition evaluation method according to claim 1, wherein the at least one sport cognitive aspect includes at least an inhibition control aspect and a semantics aspect.

3. The cognition evaluation method according to claim 1, wherein evaluating the sport cognition level of the at least one sport cognitive aspect according to the cognition task outcome comprises:
establishing a normal distribution model with a plurality of general cognition task outcomes, wherein the normal distribution model has a plurality of value domains, and each of the value domains corresponds to a level; and
using the level at which the cognition task outcome corresponding to the at least one sport cognitive aspect is located as the sport cognition level.

4. The cognition evaluation method according to claim 1, wherein after evaluating the sport cognition level, the method further comprises:
determining whether the sport cognition level is a high sport cognition level or a low sport cognition level;
outputting a high intensity training suggestion when determining the sport cognition level is the high sport cognition level; and
outputting a low intensity training suggestion when determining the sport cognition level is the low sport cognition level.

5. The cognition evaluation method according to claim 3, wherein the level includes a high sport cognition level, and establishing the normal distribution model comprises:
adjusting a boundary value of the high sport cognition level according to a sport scale.

6. The cognition evaluation method according to claim 3, wherein after using the level at which the cognition task outcome corresponding to the at least one sport cognitive aspect is located as the sport cognition level, the method further comprises:
updating a boundary value of the level at which the cognition task outcome is located using the cognition task outcome.

7. The cognition evaluation method according to claim 1, wherein the cognitive aspects comprise: an inhibition control aspect, a visual perception aspect, a visual attention aspect, a memory aspect, an emotion aspect and a semantics aspect.

8. A cognition evaluation system, comprising:
a brain state measuring instrument, configured to obtain brain physiology information associated with a subject; and
a computing device, configured to obtain a plurality of cognitive aspects according to the brain physiology information, and select at least one of the cognitive aspects according to a sport type as at least one sport cognitive aspect, the computing device configured to obtain a cognition task outcome corresponding to the at least one sport cognitive aspect, and further evaluate and outputs a sport cognition level of the at least one sport cognitive aspect according to the cognition task outcome.

9. The cognition evaluation system according to claim 8, wherein the at least one sport cognitive aspect includes at least an inhibition control aspect and a semantics aspect.

10. The cognition evaluation system according to claim 8, wherein the computing device evaluates the sport cognition level of the at least one sport cognitive aspect according to the cognition task outcome by:
establishing a normal distribution model with a plurality of general cognition task outcomes, wherein the normal distribution model has a plurality of value domains, and each of the value domains corresponds to a level, and
using the level at which the cognition task outcome corresponding to the at least one sport cognitive aspect is located as the sport cognition level.

11. The cognition evaluation system according to claim 8, wherein the computing device further outputs a high intensity training suggestion when the computing device determines the sport cognition level is a high sport cognition level, and outputs a low intensity training suggestion when the computing device determines the sport cognition level is a low sport cognition level.

12. The cognition evaluation system according to claim 10, wherein the level includes a high sport cognition level, and establishing the normal distribution model performed by the computing device comprises: adjusting a boundary value of the high sport cognition level according to a sport scale by the computing device.

13. The cognition evaluation system according to claim 10, wherein after the computing device uses the level at which the at least one sport cognitive aspect is located as the sport cognition level, the computing device further updates a boundary value of the level at which the sport cognition level is located using the sport cognition level.

14. The cognition evaluation system according to claim 8, wherein the cognitive aspects comprises: an inhibition control aspect, a visual perception aspect, a visual attention aspect, a memory aspect, an emotion aspect and a semantics aspect.
